(19) United States
(12) Patent Application Publication
(10) Pub. No.: US 2012/0053675 A1
(43) Pub. Date: Mar. 1, 2012

(54) MEDICAL IMPLANT, PARTICULARLY VALVE IMPLANT, FOR IMPLANTATION IN AN ANIMAL AND/OR HUMAN BODY AND METHOD, PARTICULARLY PRODUCTION METHOD, FOR PRODUCING AN IMPLANTATION APPARATUS FOR THE MEDICAL IMPLANT

(75) Inventor: Alexander Borck, Aurachtal (DE)

(73) Assignee: BIOTRONIK AG, Buelach (CH)

(21) Appl. No.: 13/206,319

(22) Filed: Aug. 9, 2011

Related U.S. Application Data
(60) Provisional application No. 61/378,422, filed on Aug. 31, 2010.

(51) Int. Cl. A61F 2/82; B23P 11/00

(52) U.S. Cl. 623/1.24; 29/428

(57) ABSTRACT

A medical implant (10), and particularly to a valve implant (12), for implantation in an animal and/or human body (14), comprising a base body (16) which can be expanded at least in some regions. It is proposed that the base body (16) which can be expanded at least in some regions comprises a first actively expandable region (18) and at least one second passively expandable region (20, 22).

### CROSS REFERENCE TO RELATED APPLICATIONS

This patent application claims the benefit of U.S. Provisional Patent Application No. 61/378,422, filed on Aug. 31, 2010, which is hereby incorporated by reference in its entirety.

### TECHNICAL FIELD

The invention relates to a medical implant, and particularly to a valve implant, for implantation in an animal and/or human body, and to a method, and particularly to a production method, for producing an implantation apparatus for the medical implant.

### BACKGROUND

In medicine, implants are used which are introduced permanently or at least for an extended period into an animal and/or human body so as to fulfill replacement functions. For example valve implants are known, such as aortic valve implants which assume the function of the natural aortic valve. After expanding the implant structure, the valve implant is fixed in place immediately after implantation and assumes the position of the natural aortic valve.

A frequent problem is that the implant is fixed in a malposition, which can result in failure of the implant. This is frequently the case, for example, with calcification, that is the deposition of calcium salts, and particularly calcium phosphate (hydroxyapatite), at the structures of the heart, and notably with highly asymmetrically calcified aortic stenosis.

### SUMMARY

It is the object of the invention to create a medical implant which can be implanted exactly and reliably at an implantation site.

The invention relates to a medical implant, and particularly to a valve implant, for implantation in an animal and/or human body, having a base body which can be expanded at least in some regions.

It is proposed that the base body which can be expanded at least in some regions comprises a first actively expandable region and at least one second passively expandable region. The embodiment according to the invention can provide an implant which can be optimally positioned and anchored well. Furthermore, advantageously it can be adapted to the parameters and/or anatomic circumstances of an implantation site, such as a calcification on a blood vessel wall and/or an is annulus, and/or another congenital and/or pathological anomaly of the implantation site. Furthermore a pressure gradient of a flow medium acting on the medical implant, such as blood, can be kept homogeneous, which advantageously results in lower material stress of the base body of the implant and thereby in lower fatigue risk, particularly in the case of nitinol base bodies, given the uniform opening of the base body. This in turn results in a long service life of the valvular cusps and therefore of the valve. In addition, given the improved functionality of the valve, which can thus withstand a higher pressure gradient, better clinical results can be achieved for an asymmetrically calcified annulus compared to the conventional valve implants. Because of the embodiment according to the invention, in addition the symmetry of the flow dynamics of the flow medium can be increased, which advantageously reduces any further calcification risk.

In this context, an “implant” shall be understood in particular as a body which permanently or for an extended period fulfills at least one replacement function upon implantation in an animal and/or human body. Conceivable are all medical implants that appear expedient to the person skilled in the art, for example a cardiac pacemaker, a brain pacemaker, a cardiac implant, a cochlear implant, a retina implant, a dental implant, an implant for joint replacement, a vascular prosthesis, or particularly advantageously a design of the medical implant as a valve implant is proposed. A “valve implant” shall be understood in particular as a body which permanently or for an extended period fulfills at least one replacement function of a check valve upon implantation. Conceivable are all medical valve implants that appear expedient to the person skilled in the art, for example an aortic valve, a pulmonary valve, a mitral valve or a tricuspid valve implant, or particularly advantageously a design of the medical implant as a stent is proposed, and particularly as a coronary stent, having an implant structure that is reversibly or irreversibly connected to the stent. In this context, an “implant structure” shall be understood in particular as an aortic valve, a pulmonary valve, a mitral valve, or a tricuspid valve made of natural and/or synthetic materials. In general, however, any other implant structure that appears useful to the person skilled in the art would be conceivable. Because of the implementation of the implant as a stent, or because the base body comprises a stent, a structure can be provided which is easy to implant in terms of the design thereof.

Furthermore, in this context a “base body” shall be understood in particular as a structure, for is example a wire mesh, which substantially forms a configuration and/or a shape of the valve implant, particularly a shape of the stent and/or the stent as such. In addition, the base body is preferably produced from an elastic or super elastic material, such as a metallic material and/or a combination of multiple metallic materials, for example iron, magnesium, nickel, tungsten, titanium, zirconium, niobium, tantalum, zinc, silicon, lithium, sodium, potassium, calcium, manganese and/or any other material that may appear useful to the person skilled in the art. A zinccalcium alloy would also be possible. The term “actively expandable” shall be understood in particular to mean that the region expands or can be expanded independently or automatically, this being without external help. Advantageously, the actively expandable region is produced from a shape memory material, such as a copper-zinc-aluminum alloy and/or a nickel-titanium alloy, preferably nitinol. In addition, it would also be conceivable to provide two actively expandable regions, which have the same or different expansion characteristics. The term “passively expandable” shall be understood in particular to mean that the region can be expanded or deformed not independently and/or by way of a force that is applied from the outside. This shall in particular not be understood as an expansion using a self-expandable material and/or by the first region. In a simple design, the passive expansion can be carried out using a balloon catheter. It may furthermore be advantageous for the passively expandable region and/or the base body to comprise at least cobalt and/or chromium, preferably in the form of stainless steel or medical stainless steel and/or a Cr—Ni—Fe steel—in this case preferably the alloy 316L—or a Co—Cr steel. With this embodiment, an implant can be provided which produces satisfactory coating results and has good dilating properties and advantageous flexibility, combined with high stability. In principle, however, it is also conceivable for the base body of the valve implant to be made at least partially of plastic, a ceramic and/or a biodegradable material. Given the two unevenly expandable regions, advantageously different expansion mechanisms can be employed, which results in an implant that is especially versatile in terms of the use thereof.

It is furthermore proposed that the second region can be expanded temporally after the first region. The term “temporally after” shall be understood in particular as subsequent to and/or with a time delay of more than 10 seconds, preferably more than 60 seconds, and particularly preferably more than 300 seconds, between the active and the passive expansion. By implementing the time delay, the positioning can be improved, whereby advantageously malpositions of the implant after pre-implantation by the actively expandable region can be compensated for by way of the passive region. In this way, failure of the implant can be prevented with a simple design.

Advantageously, the first region is provided to widen the second region in a transversal direction during an expansion of at least the first region. In this context, a “transversal direction” shall be understood in particular as a direction extending from a geometric center of gravity of an area fictitiously spanned by the first region toward the outside. The transversal direction is in particular a radial direction, and a “geometric center of gravity of an area” means in particular a center, and particularly advantageously a center of a circle of an area, in particular a circular area, having a maximum extension in the implanted state of the implant substantially perpendicular to the flow direction of the flow medium. In this way, the implant structure or the valve is received or can be received with accurate fit in an inside cross-section of the implant. The term “widen” here defines in particular an augmentation of a cross-section of a fictitious area spanned by the second region. Due to the widening, an impairment of the blood flow by the second region can be effectively prevented in the pre-implanted state of the implant.

To this end, the first actively expandable region and the second passively expandable region can be configured in any arbitrary arrangement relative to each other that appears expedient to the person skilled in the art, for example behind each other and/or in the transversal direction on top of each other. Advantageously, compared to the first region, at least a portion of the second region, starting from the geometric center of gravity or center of the fictitious area spanned by the first region, has a larger distance from the center of gravity/center than a distance of the first region from the center of gravity/center, or the second region is disposed further to the outside in the radial direction than the first region, and/or the second region is disposed above the first region. Furthermore, the second region is disposed in the circumferential direction around the first actively expandable region. Furthermore, the second region preferably extends around the entire circumference of the first region. In principle, however, an extension over sections of the circumference would also be conceivable.

Widening of the second region in a simple design can be achieved if the second region is intert-wined with the first region over a region extending in an axial direction. To this end, the term “intertwined” shall be understood in particular as woven, knitted, wrapped, linked, knotted, interlaced or any other synonym that appears useful to the person skilled in the art. The connection between the two regions is advantageously flexible or not rigid, so that a relative movement in the axial direction and/or in the circumferential direction can take place between the two regions.

Furthermore, it is advantageous for the actively expandable region to be preferably composed of cells. These cells can have any shape that appears expedient to the person skilled in the art, such as round, oval, triangular, rectangular and/or rhombic. This shape is particularly designed so as to be foldable. In a particularly preferred embodiment, the cells have a substantially rhombic configuration. The term “substantially rhombic” here shall be understood in particular such that shapes that are similar to a rhomb or rhombus, for example a rhombic shape having rounded edges and/or concave and/or convex sides, are also covered by the term “rhombic”. Furthermore, the second passively expandable region may also be composed of cells having one and/or more of the above shapes and properties. With this shape, a particularly stable base frame of the implant can be provided.

In a further embodiment of the invention, it is proposed that the base body can be shortened in the axial length thereof by way of an expansion. This can be done in a particularly simple design, for example, by way of the rhombic cells. Due to the embodiment according to the invention, prior to the expansion, a greater length of the implant compared with non-shortenable implants can advantageously be used to accommodate an implant structure or a valve. Furthermore, in this way a crimping radius can be advantageously minimized when producing an implant apparatus having the implant according to the invention.

Furthermore it is proposed that the second region is provided so as to compensate for a difference in the shape of the inside cross-section of the base body and a cross-sectional surface of the implantation side. The term “provided” shall be understood in particular as specially equipped, designed and/or prepared. In this context, a “shape of the inside cross-section of the base body” shall be understood in particular as a largely round or cylindrical shape, so that the cusps of the valve can open and close without difficulty. A “cross-sectional surface of an implantation site” in this case shall be understood in particular as a highly asymmetrical or non-circular site, and particular one having a calcified aortic stenosis. As a result, the second region advantageously adapts the non-uniform shapes of the outside diameter of the valve or of the inside cross-section of the base body to the cross-sectional surface of the implantation site, whereby the implant notably takes the local circumstances of the implantation site into consideration. In this way, advantageously non-symmetry of the blood vessel wall or of the annulus can be compensated for, and still a largely round, symmetrical inside shape of the base body for the necessary trouble-free and complication-free functioning of the cusps of the valve can be preserved.

Furthermore, it may be advantageous for the medical implant to have a separating means, which separates the first region from the second region. In this context, a “separating means” shall be understood in particular as any means that appears expedient to the person skilled in the art, such as a spacer and/or in particular a coating, wherein a “coating” shall be understood in particular as an at least partial, and preferably a complete, sheathing of the first and/or second regions or the struts thereof or stent struts. The coating is particularly advantageously formed by an amorphous silicon carbide. In general, however, any other coating made of insulating or semiconductor materials that appears suitable to the person skilled in the art would be conceivable, which effectively prevents contact between the materials of the two regions, and notably contact between the metallic materials such as NiTi or CoCr in the presence of electrolytes. The coating allows a contact problem of the different noble metals of the two regions to be solved using a simple design and in a space-saving manner.

In addition, it is proposed that the implant comprises at least one anchoring means, whereby the implant can be fixed particularly conveniently. In a particularly advantageous embodiment, the anchoring means is disposed on the second region and in the intended final state or in the implanted state has at least one extremal point, which starting from the geometric center of gravity/center of the area spanned by the first region has a larger distance from the center of gravity/center than a distance of the first region from the center of gravity/center. In this context, an “anchoring means” shall be understood in particular as a loop, a hook, a tip and/or another means considered suited by the person skilled in the art. An “intended final state” here shall be understood in particular as an implanted state of the implant at the implantation site, such as a site of a defective heart valve and/or an annulus. To this end, the implant or the stent having the two regions is expanded and anchored at the implantation site in the correct position. Furthermore, the term “extremal point” shall be understood in particular as a maximum in the extension of an outer cross-section, starting from the geometric center of the base body toward the outside, and in the direction of a wall of a blood vessel in the implanted state. By implementing the extremal point and the larger distance, the implant can advantageously be fixed at predetermined points.

The anchoring means can additionally be moved particularly advantageously independently from the remaining second region, wherein “can be moved” shall be understood in particular as radially movable, and particularly advantageously radially movable in the direction of the wall of the blood vessel, such as the aortic wall, during the expansion by way of an expansion region of an expansion means, such as a balloon catheter. The anchoring means preferably has a lower retaining force than the remaining second region, wherein a “retaining force” here shall be understood in particular as a force, the vector of which points in the direction of the geometric center of gravity or the center of a circle of the base body. The lower retaining force is caused, for example, by a reduction of contact sites of the anchoring means with the remaining second region with respect to structures or cells of the second region. Because of the lower retaining force, during the expansion by way of the expansion region the anchoring means has a greater ability than the remaining second region to be moved radially outwardly, this being in the direction of the wall of the blood vessel. This difference in retaining forces is independent of any resistance acting from the outside, such as the force of the wall of the blood vessel (aorta). Consequently, the ancboring means can be disposed obliquely relative to a flow direction of the flow medium. Advantageously, a variable outside contour of the stent can be adjusted in the implanted state by way of the anchoring means. In addition, good anchoring can be provided by the expansion means with advantageously low force expenditure.

Particularly exact positioning and fixation can advantageously be achieved when the base body has at least two second regions. In principle, the implant may also comprise an arbitrary number of second passively expandable regions. In the version comprising two second regions, advantageously a double balloon catheter is used as the expansion means, and a segmented balloon catheter is used in the case of more than two second regions. Preferably at least one first region is disposed axially between two second regions.

According to a preferred refinement, the implant has a valve, which is disposed in an axial direction at least between two second regions or between two subsequently expandable regions. To this end, the valve can be connected to the base body or to the first region by way of any connecting mode considered useful by the person skilled in the art, for example by sewing and/or gluing. With this structure, an implant is provided which in the implanted state can assume an advantageously waisted shape, which is advantageous for automatically finding/automatic positioning in the optimal position.

According to a further embodiment of the invention, in the intended final state the at least two second regions are disposed axially in front of and behind an annulus in the flow direction of the flow medium. A “flow direction of a flow medium” here shall be understood in particular as the scientifically known flow direction of arterial and/or venous blood in the heart, and particularly advantageously in the case of the aortic valve the flow of blood from the left ventricle into the aorta. The annulus is preferably the aortic annulus. Through the implementation of the structure according to the invention, the implant can be adapted particularly well to the anatomy of the heart or a heart valve region having an aortic bulbus, for example, notably in the case of the waisted shape.

An embodiment of the medical implant as an aortic valve is particularly advantageous, whereby a sophisticated replacement structure for the heart valve most frequently subject to malfunctions can be provided. In addition, complications such as mitral valve dysfunctions or the necessity of a pacemaker can advantageously be reduced. A design as a pulmonary valve or a design as a mitral valve is also conceivable.

Advantageously, a deposit-inhibiting, and particularly a calcification-inhibiting coating may be provided on the implant, notably homocysteine acid. In this way, the risk of a dysfunction or a functional failure of the valve implant can be further reduced.

The invention furthermore relates to a method, and particularly to a production method, for producing an implantation apparatus for a medical implant, comprising a base body having a first actively expandable region and a second passively expandable region.

It is proposed that the base body be mounted on an expansion means, wherein at least the second region of the base body is disposed in the circumferential direction around an expansion region of the expansion means, and the resulting implantation complex is combined with a retaining means. In this context, an “implantation apparatus” shall be understood in particular as an apparatus by means of which an implantation is carried out and which comprises the implant. An “implantation complex” shall be understood in particular as a construct that is composed of the implant and a further part, such as the expansion means or the balloon catheter. A “retaining means” here shall denote in particular a means such as a hose cover known to the person skilled in the art, which prevents active expansion of the first region. The implant is preferably connected to the expansion means by way of a reversible connection type, wherein any connection type that appears suitable to the person skilled in the art, such as crimping, may be used. With the embodiment according to the invention, an implantation apparatus can be provided which advantageously combines two expansion mechanisms.

Using the implant described above and the implantation apparatus described, a method, and particularly an implantation method, for implanting the medical implant in an animal and/or human body by way of the implantation apparatus can be carried out, wherein in a first step the implantation complex is released from the retaining means so as to expand the first region of the base body of the implant and pre-position the implant, and in a second step the second region of the base body of the implant is expanded by way of the expansion region of the expansion means for final positioning and anchoring at the implantation site.

### DESCRIPTION OF THE DRAWINGS

The invention will be described in more detail hereinafter by way of example based on an embodiment illustrated in the drawings. In the drawings:

FIG. 1 is a cut-out of a medical implant according to the invention in a perspective view,

FIG. 2a is a cell of the implant of FIG. 1 in the folded state,

FIG. 2b is the cell of FIG. 2a in the expanded state,

FIG. 3 is the implant of FIG. 1 in a schematic illustration comprising two passively expandable regions,

FIG. 4 is the implant of FIG. 3 in the implanted state of an aorta,

FIG. 5 is a section IV-IV through the aortic wall having an implanted implant according to FIG. 4,

FIG. 6 is a schematic illustration of a production of an implantation apparatus cornprising an implant according to the invention, and

FIG. 7 is a schematic illustration of the insertion of the implantation apparatus according to FIG. 6 at an implantation site.

### DETAILED DESCRIPTION

In the figures, functionally equivalent or equivalently acting elements are denoted with the same reference numerals. The figures are schematic illustrations of the invention. They depict non-specific parameters of the invention. In addition, the figures only reflect typical embodiments of the invention and are not intended to limit the invention to the embodiments that are illustrated.

So as to avoid unnecessary repetitions, elements in a figure that are not described in detail are provided with a reference to the respective description of the elements in the preceding figures.

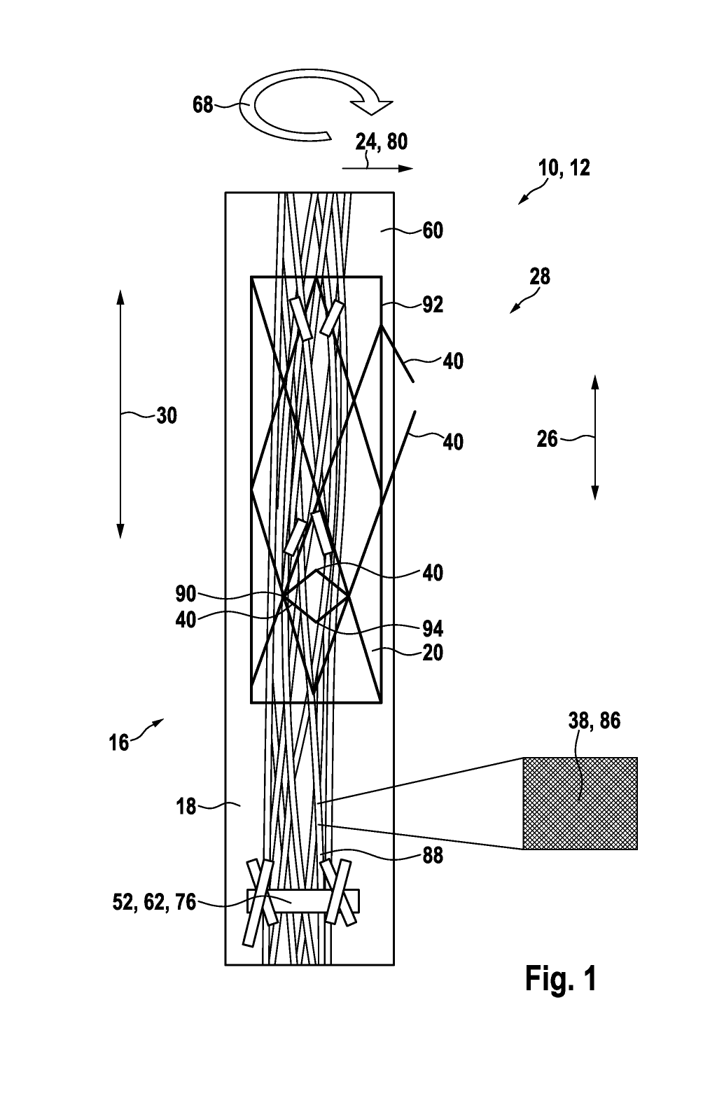

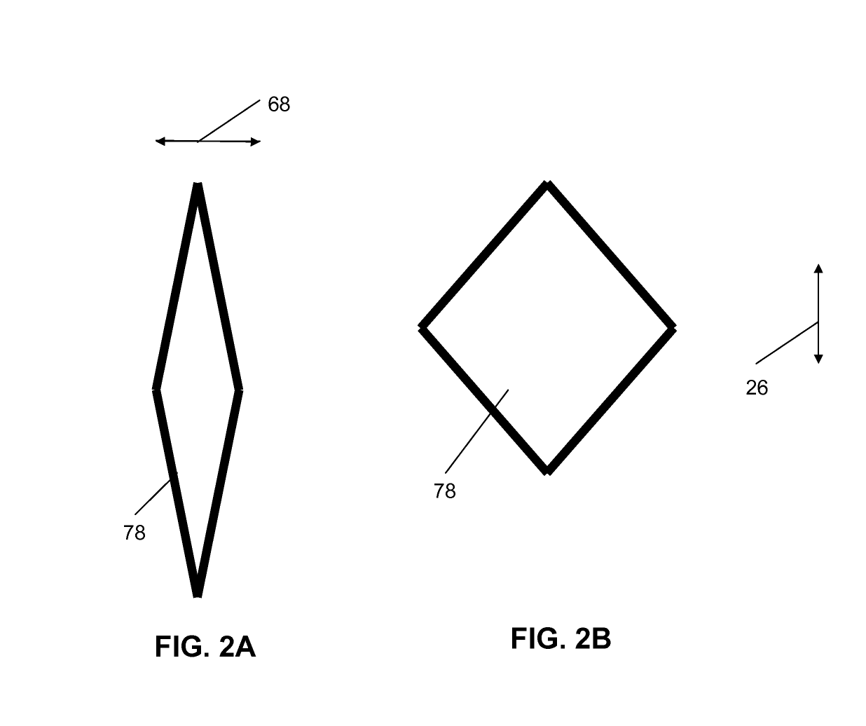

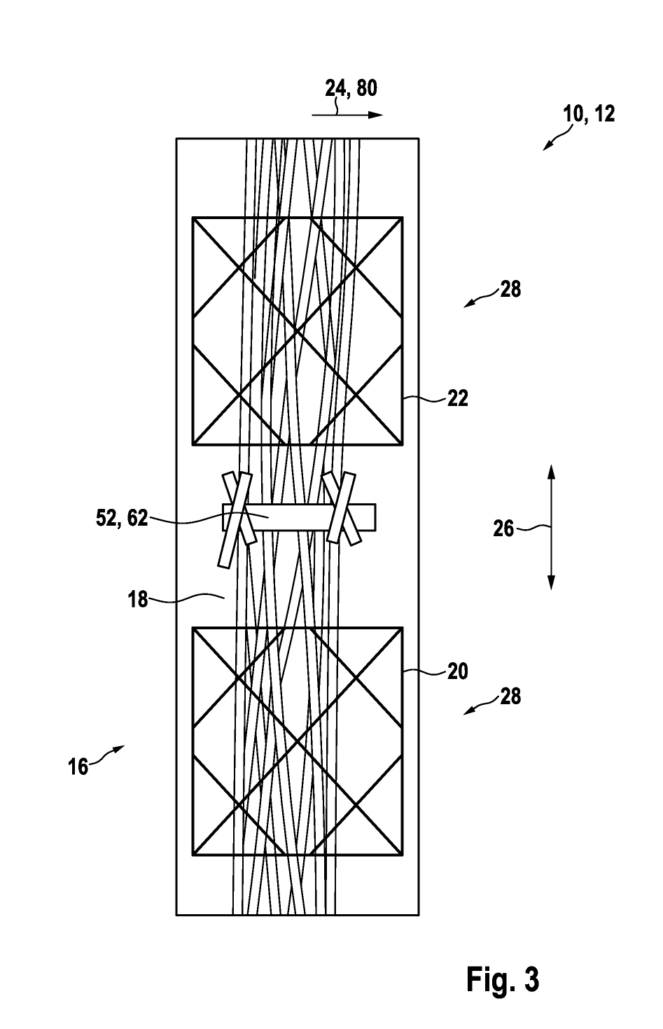

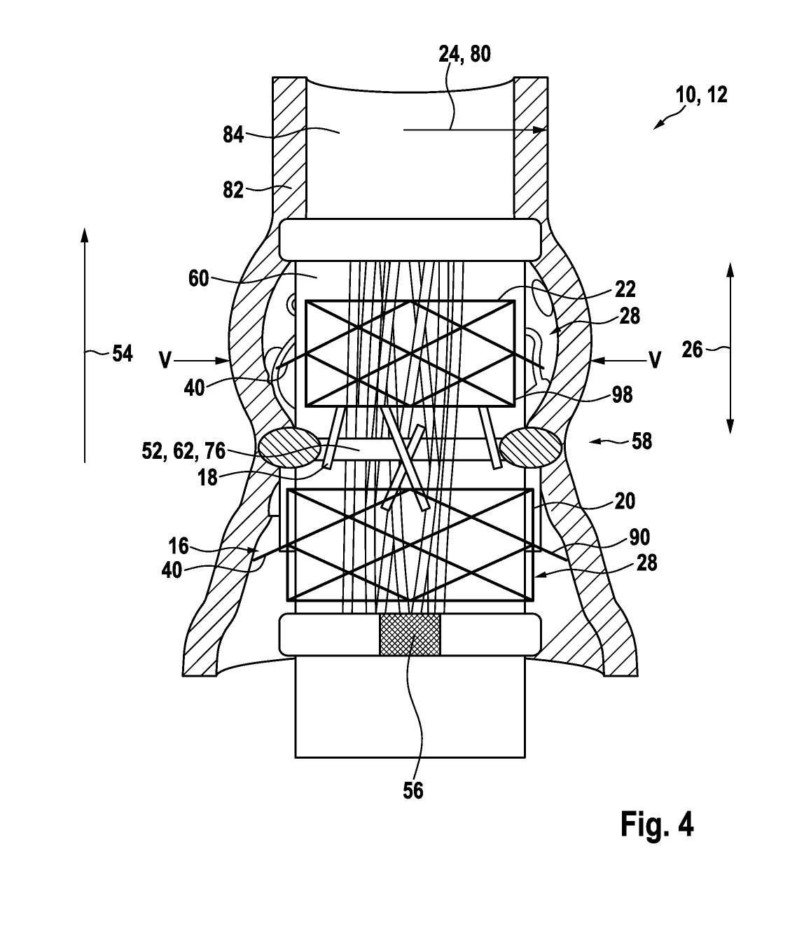

FIG. 1 shows part of a medical implant 10 or of a valve implant 12 for implantation in an animal and/or human body 14, comprising a base body 16 that can be expanded in some regions and includes a self-expanding stent 60. The implant 10 is furthermore an aortic implant, in which an implant structure 76 configured as an artificial percutaneous aortic valve 62 is fastened to the base body 16 or to the stent 60. The base body structure of the base body 16 is a wire mesh, which is formed by a first actively expandable region 18 and made of nitinol. The wire mesh and/or the base body 16 comprise a plurality of cells 78, which have a substantially rhombic shape and are disposed next to each other in the circumferential direction 68 and behind each other in the axial direction 26. Due to this embodiment, the implant 10 can be folded, and during automatic expansion the cells 78 widen in the circumferential direction 68, whereby the extensions thereof in the axial direction 26 are shortened, as is shown by way of example for one cell 78 in FIGS. 2a and 2b. In this way, the base body 16 can also be shortened in the axial length 30 thereof by way of the expansion, as is apparent in particular from a comparison of the illustrations of the implant 10 in FIGS. 3 and 4.

The base body 16 furthermore comprises a second passively expandable region 20, which is made of medical stainless steel comprising at least cobalt and/or chromium. In addition, analogous to the first region 18, the second region 20 comprises a plurality of rhombic cells 78. The second region 20 is intertwined with the first region 18 over a region 28 extending in the axial direction 26, wherein the intertwined structure can be configured such that a relative movement between the regions 18, 20 is possible. For reasons of clarity, the intertwined structure of the regions 18, 20 is shown only by way of example in FIG. 1.

Given this arrangement of the regions 18, 20 relative to each other, the first region 18 is provided to widen, during the expansion thereof, the second region 20 in a transversal direction 24 or in a radial direction 80, that is, to widen it in the intended final state or in the implanted state of the implant 10 toward a wall 82 of a blood vessel 84, such as an aorta.

In addition, the implant 10 has a separating means 38, which separates the first region 18 from the second region 20. This separating means 38 is formed by a coating 86 made of amorphous silicon carbide and is applied to the first actively expandable region 18 of the base body 16 or of the stent struts 88 thereof (shown symbolically in FIG. 1).

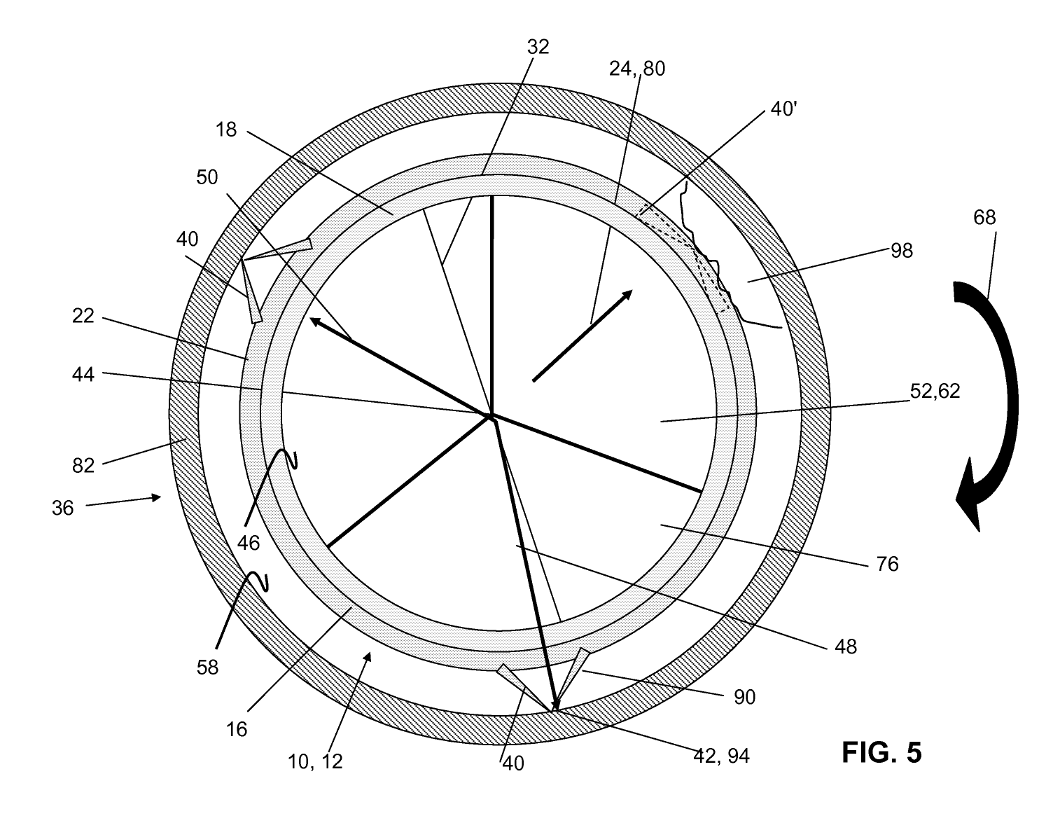

Furthermore, the implant 10 or the base body 16 comprises a plurality of anchoring means 40 on the second region 20. These anchoring means 40 are formed by struts 90 that are disposed at an angle and therefore have a V shape. An end of a strut 90 that is not connected to the other strut 90 is integrally formed on an intersecting point 92 of a cell 78 of the second region 20. As a resuit, a fastening site to the second region 20 is lacking at a connecting site 94 of the struts 90 or at a tip of the V, whereby the anchoring means 40 has a lower retaining force than the cells 78 of the second region 20. In this way, the anchoring means 40 can be moved relative to the second region 20 or can be moved in the radial direction 80 and can be disposed relative to a flow direction 54 of a flow medium 56, such as blood, obliquely at a maximum angle of 45°. When the implant 10 or the second region 20 is widened with a balloon catheter, the anchoring means 40 can be pushed radially further to the outside or toward an aortic wall. In this way, an anchoring means 40 has an extremal point 42 at the connecting site 94 of the struts 90 in the intended final state. Starting from a geometric center of gravity 44 or a center of a circle of an area 46 spanned by the first region 18, the extremal point has a larger distance 48 from the center of gravity 44 than a distance 50 of the first region 18 from the center of gravity 44 (see FIG. 5).

FIG. 3 shows the complete implant 10. The base body 16 comprises two second regions 20, 22, wherein the second second region 22 is configured analogous to the first second region 20 in terms of design and function. Furthermore, a valve 52 is disposed in the axial direction 26 between the two second regions 20, 22.

FIG. 4 shows a schematic view of the medical implant 10 in the implanted form, in an annulus 58 of a natural aortic valve, for example, which is disposed in the blood vessel 84 or the aorta, in front of a left ventricle 96 of the heart. To this end, the first second region 20 is disposed in front of the annulus 58 in the flow direction 54 and the second second region 20 is disposed behind the annulus 58.

When the implant 10 is implanted, the implant 10 is pre-positioned as a result of the automatic expansion of the actively expandable region 18 of the base body 16. Here, in particular anomalies at the annulus 58 or calcifications 98 have no significant effect on the position of the implant 10. The second regions 20, 22 are expanded temporally after the first region 18. Depending on the circumstances of an implantation site 36, repositioning may take place, or the implant 10 may assume an optimal fit, and the implant 10 is fixed in place as a result of the press fit of the second regions 20, 22 with the wall 82 or the movement of the anchorings 40 in the direction of the wall 82 of the blood vessel 84.

If calcifications 98 are present at the wall 82 of the blood vessel 84, the second regions 20, 22 move until they come in contact with the calcification 98. In addition, it is prevented, for example, that anchoring means 40′ can be pushed to the outside, as is shown in FIG. 5 illustrating a section through the aortic wall with a view onto the implant 10. As a result, the second region 20 and/or the anchoring means 40 are provided so as to compensate for a difference in the shape of an inside cross-section 32 of the base body 16 and a cross-sectional surface 34 of the implantation site 36.

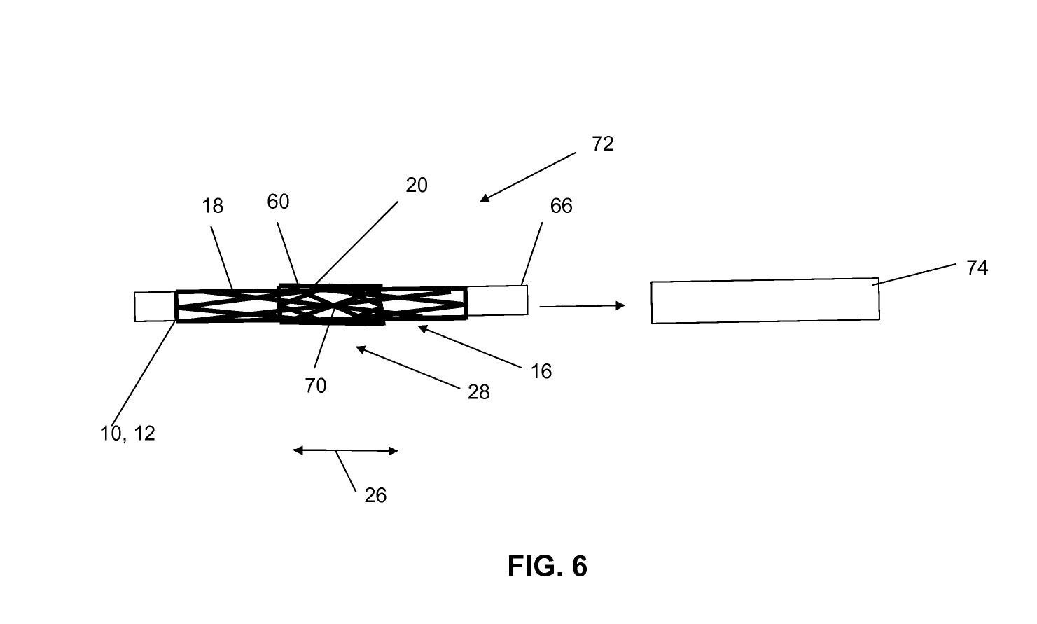

FIG. 6 is a schematic illustration of a production of an implantation apparatus 64 for the medical implant 10. To this end, the base body 16 is mounted on an expansion means 66 or fastened by a crimping process at −78° C. The second region 20 of the base body 16 is disposed in the circumferential direction 68 around an expansion region 70 of the expansion means 66, for example a balloon region of a balloon catheter (analogous to region 22 for a double balloon catheter). Thereafter, the resulting implantation complex 72 comprising the implant 10 and expansion means 66 is combined with the retaining means 74 or the implantation complex 72 is inserted into the retaining means 74.

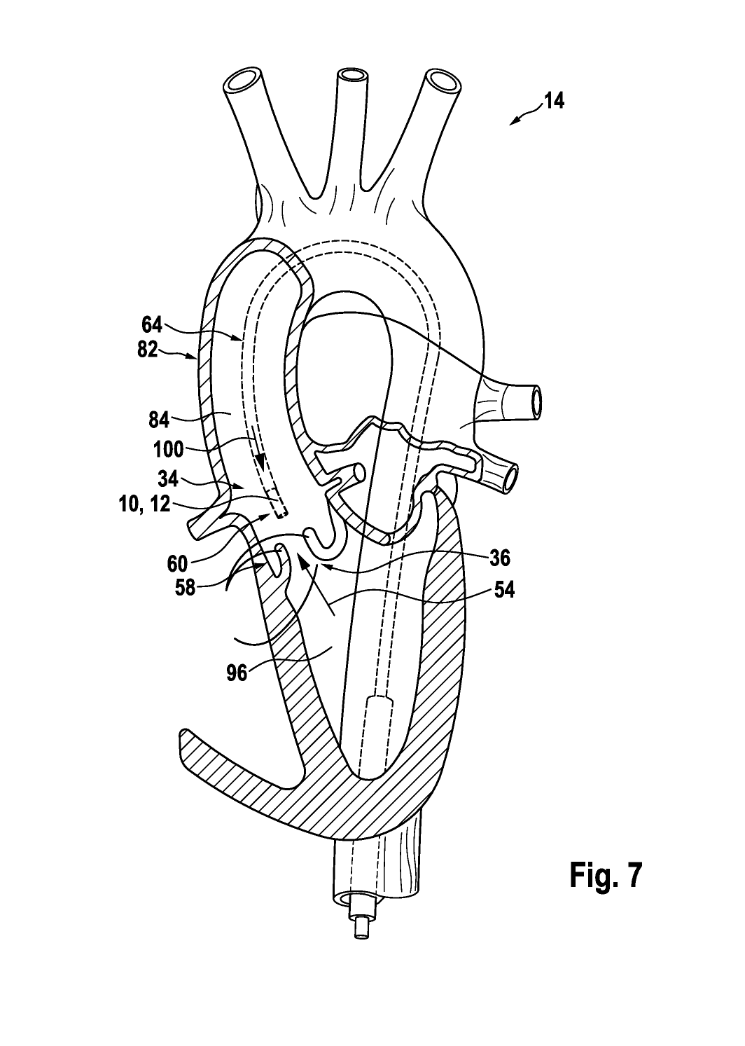

FIG. 7 schematically illustrates the insertion of the medical implant 10 in a partial section view. The implantation complex 72 comprising the balloon catheter and implant 10 is fed in the known manner to the implantation site 36, such as the annulus 58 of the natural aortic valve with cusps. Here, an implantation direction 100 is counter to the flow direction 54. Once the correct position has been reached, the implantation complex 72 is released from the retaining means 74, whereby the first actively expandable region 18 automatically expands and pre-positions the implant 10. In a second temporally subsequent step, the second region 20 of the base body 16 is passively expanded by means of the expansion region 64 of the expansion means 60. This results in the final positioning and anchoring of the implant 10 at the implantation site 36.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NUMERALS

10 Implant
12 Valve implant
14 Body
16 Base body
18 Region
20 Region
22 Region
24 Direction
26 Direction
28 Region
30 Length
32 Inside cross-section
34 Cross-sectional surface
36 Implantation site
38 Separating means
40 Anchoring means
42 Extremal point
44 Center of gravity
46 Area
48 Distance
50 Distance
52 Valve
54 Flow direction
56 Flow medium
58 Annulus
60 Stent
62 Aortic valve
64 Implantation apparatus
66 Expansion means
68 Circumferential direction
70 Expansion region
72 Implantation complex
74 Retaining means
76 Implant structure
78 Cell
80 Direction
82 Wall
84 Blood vessel
86 Coating
88 Stent strut
90 Strut
92 Intersecting point
94 Connecting site
96 Ventricle
98 Calcification
100 Implantation direction

What is claimed is:

1. A medical implant, and particularly a valve implant, for implantation in an animal and/or human body, comprising a base body that can be expanded at least in some regions, characterized in that the base body which can be expanded at least in some regions has a first actively expandable region and at least one second passively expandable region.

2. The medical implant according to claim 1, characterized in that the second region can be expanded temporally after the first region.

3. The medical implant according to claim 1, characterized in that the first region is provided to widen the second region in a transversal direction upon an expansion of at least the first region.

4. A medical implant according to claim 1, characterized in that the second region is intert-wined with the first region over a region extending in an axial direction.

5. A medical implant according to claim 1, characterized in that the base body can be shortened in the axial length thereof by way of an expansion.

6. A medical implant according to claim 1, characterized in that the second region is provided to compensate for a difference between a shape of an inside cross-section of the base body and a cross-sectional surface of an implantation site.

7. A medical implant according to claim 1, characterized by a separating means, which separates the first region from the second region.

8. A medical implant according to claim 1, characterized by at least one anchoring means of at least the second region, which in an intended final state has at least one extremal point, which starting from a geometric center of gravity of an area spanned by the first region has a larger distance from the center of gravity than a distance of the first region from the center of gravity.

9. A medical implant according to claim 1, characterized in that the base body has at least two second regions.

10. A medical implant according to claim 9, characterized by a valve, which is disposed in an axial direction at least between the at least two second regions.

11. A medical implant according to claim 9, characterized in that in an intended final state the at least two second regions are disposed axially in front of and behind an annulus in the flow direction of a flow medium.

12. A medical implant according to claim 9, characterized in that the second region of the base body comprises at least cobalt and/or chromium.

13. A medical implant according to claim 1, characterized in that the base body comprises a stent.

14. A medical implant according to claim 1, characterized in that the separating means comprises amorphous silicon carbide.

15. A medical implant according to claim 1, characterized by an embodiment as an aortic valve.

16. A method, and particularly a production method, for producing an implantation apparatus for a medical implant, comprising a base body having a first actively expandable region and a second passively expandable region at least according to claim 1, characterized in that the base body is mounted on an expansion means, wherein at least the second region of the base body is disposed in a circumferential direction around an expansion region of the expansion means and a resulting implantation complex is combined with a retaining means.
